(19) United States
(12) Patent Application Publication
(10) Pub. No.: US 2015/0359763 A1
(43) Pub. Date: Dec. 17, 2015

(54) USE OF 3-BROMOPYRUVATE AS A CONTRACEPTIVE

(71) Applicant: Wei YAN, Reno, NV (US)

(72) Inventor: Wei Yan, Reno, NV (US)

(73) Assignee: Board of Regents of the Nevada System of Higher Education, on behalf of the University of Nevada, Reno, NV (US)

(21) Appl. No.: 14/764,526

(22) PCT Filed: Jan. 28, 2014
(86) PCT No.: PCT/US14/13288

§ 371 Date: Jul. 29, 2015

Related U.S. Application Data
(60) Provisional application No. 61/758,711, filed on Jan. 30, 2013.

(51) Int. Cl. A61K 31/19; A61K 45/06; A61F 6/04; A61K 9/00

(52) U.S. Cl. CPC A61K 31/19; A61K 9/0034; A61K 9/0014; A61K 45/06; A61F 6/04

(57) ABSTRACT

Disclosed herein are methods of inhibiting sperm motility. In some embodiments, a method of inhibiting sperm motility includes contacting a sperm with an effective amount of 3BP, thereby inhibiting sperm motility. Also disclosed herein are methods of contraception. In some embodiments, a method of contraception includes administering an effective amount of 3BP to the vagina so that sperm in contact with 3BP change their swimming patterns, which are incompatible with fertilization, thus preventing pregnancy. In one embodiment, a method includes topical application of 3BP to the penis in an amount effective to cause complete loss of motility of ejaculated sperm during sexual intercourse, thereby achieving contraception. In some examples, 3BP is administered as a spermicide in the forms of a jelly (gel), films, sponges, foams and timed-release mesh. In some examples, condoms are coated with 3BP-containing lubricant to achieve contraception.

### CROSS REFERENCE TO RELATED APPLICATION

This application claims the benefit of U.S. Provisional Application No. 61/758,711, filed Jan. 30, 2013, which is herein incorporated by reference in its entirety.

### FIELD

This disclosure relates to contraceptive agents, and in particular, to the use of 3-bromopyruvate (3BP) to inhibit sperm motility and conception.

### BACKGROUND

Fifty percent of the pregnancies in the United States are unintended, highlighting the needs for convenient and effective contraceptive measures. Spermicide refers to a contraceptive substance that immobilizes sperm when administered intravaginally prior to intercourse and thus prevents sperm from reaching eggs to achieve contraception. As a contraceptive, spermicides may be used alone. Usually, spermicides are combined with contraceptive barrier methods such as diaphragms, condoms, cervical caps, and sponges. Combined methods are believed to result in lower pregnancy rates than either method alone. Conventional spermicides have traditionally been active compositions not specifically activated by coitus. The commonly known spermicide is nonoxynol-9 (N-9), typically administered as N-9-containing sponges that fit over the cervix, or as a lubricant applied intravaginally or on the surface of condoms. N-9 is a detergent and acts by causing breakage of the sperm plasma membrane due to its strong surfactant action. Many side effects have been associated with the repetitive application of surfactant-based spermicides, including irritation, increased incidence of sexually transmitted diseases (STD) due to cytoplasmic membrane damages and pro-inflammatory response of vagina, and increased risk of vaginal or cervical infection and irritation or ulceration. Detergent-type spermicides alter vaginal bacterial flora, and such disturbance of the vaginal microbial milieu can lead to opportunistic infections, which in turn increases the chance of HIV/STD transmission. Thus, a need exists for effective and convenient contraceptives that are not associated with these undesirable side effects.

### SUMMARY

Disclosed herein is the surprising discovery that 3-bromopyruvate (3BP) is a potent spermicide or sperm immobilizer that can cause rapid and permanent loss of sperm motility. In particular, the inventor performed a series of in vitro studies and identified that the effective concentration (EC) causing 100 percent of activated human sperm to loose motility within 20 seconds (EC₁₀₀) was 200 mM, whereas the EC₅₀ was ˜120-130 mM when pH was at 7.5 (semen pH value). At a pH value close to that of normal vaginal environment (˜pH 5.0), EC₅₀ and EC₁₀₀ of 3BP for human sperm are ˜13 mM and ˜24 mM, respectively. In less than 1 minute of exposure to 1 mM 3BP, both murine non-activated sperm (collected directly from the epididymis into physiological saline) and activated mouse or human sperm (incubated with human tubal fluid [HTF] for 30 minutes in 37° C.) became permanently immobilized with a complete loss of motility. Thus, the inventor discloses herein that 3BP is an effective contraceptive agent, such as by topical administration (e.g., vaginal or penile applications). Topical (vaginal or penile) applications expose ejaculated sperm to 3BP, causing permanent loss of motility and thus, achieving contraception.

Based on these findings, disclosed herein are methods of inhibiting sperm motility. In some embodiments, methods of inhibiting sperm motility include contacting a sperm with an effective amount of 3BP, thereby inhibiting sperm motility. In some embodiments, methods of contraception are provided. In one embodiment, a method includes administering to the vagina an amount of 3BP effective to inhibit sperm-egg fertilization, and thus prevent pregnancy. In one embodiment, a method includes topical application of 3BP to the penis in an amount effective to inhibit the motility of ejaculated sperm during sexual intercourse, thereby causing permanent loss of motility and thus, achieving contraception. In some examples, 3BP is administered as a spermicide in the form of a jelly (gel), films, sponges, foams and timed-release nanofiber or nanomesh. In some examples, condoms are coated with 3BP-containing lubricant to achieve contraception.

The foregoing and other features and advantages of the disclosure will become more apparent from the following detailed description, which proceeds with reference to the accompanying figures.

### BRIEF DESCRIPTION OF THE DRAWINGS

FIG. 1 is a bar graph illustrating the effect of various compounds known to inhibit glycolysis on sperm motility.

FIG. 2 is bar graph illustrating that the sperm-immobilizing effect of 3BP is permanent.

FIG. 3 is a graph illustrating the temporal changes in mouse sperm motility after exposure to 3BP (1 mM).

FIG. 4 is a graph illustrating 3BP analogs, methyl pyruvate (1 mM) and ethyl pyruvate (1 mM), have no effect on sperm motility in vitro.

FIG. 5 is a graph illustrating 3BP analogs, isoamyl pyruvate (1 mM) and propyl pyruvate (1 mM), have no effect on sperm motility in vitro.

FIGS. 6A and 6B each include a graph illustrating the irreversible sperm-immobilizing effects of 3BP.

FIG. 7 is a graph illustrating the sperm-immobilizing effects of 3BP on human sperm.

FIG. 8 is a graph illustrating the sperm-immobilizing effects of 3BP on human semen.

FIG. 9 is a bar graph illustrating the EC50 and EC100 of 3BP for immobilizing mouse sperm at pH 7.5.

FIG. 10 is a bar graph illustrating the EC50 and EC100 of 3BP for immobilizing mouse sperm at pH 4.8.

FIG. 11 is a bar graph illustrating the EC50 and EC100 of 3BP for immobilizing human sperm at pH 7.5.

FIG. 12 is a bar graph illustrating the EC50 and EC100 of 3BP for immobilizing human sperm at pH 5.0.

FIG. 13 is a bar graph illustrating ATP production in sperm with or without exposure to 3BP (1 mM).

FIG. 14 is a graph illustrating 3BP (1 mM) inhibition of sperm mitochondrial membrane potential.

FIG. 15 is a series of images illustrating traces of mouse sperm motility in the presence or absence of 3BP (1 mM).

FIG. 16 is a series of images illustrating traces of human sperm motility in the presence or absence of 3BP (1 mM).

FIG. 17 is a series of images illustrating no significant changes in the histology of the vagina and uterus samples after daily vaginal usage of 3BP for 3 months.

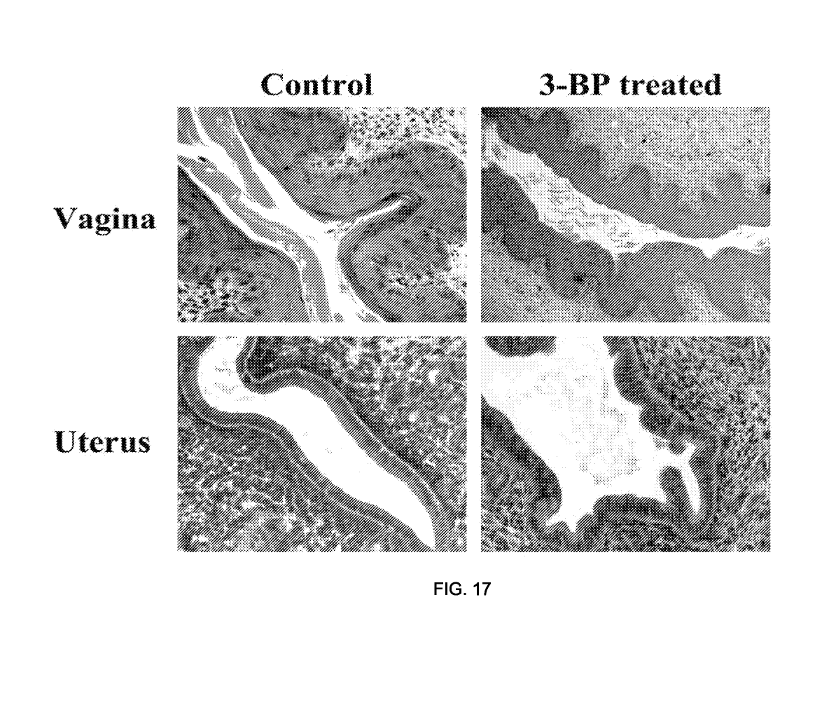

### DETAILED DESCRIPTION

Disclosed herein are methods of inhibiting sperm motility. In some embodiments, a method of inhibiting sperm motility includes contacting a sperm with an effective amount of 3BP, thereby inhibiting sperm motility. Also disclosed herein are methods of contraception. In some embodiments, a method of contraception includes administering an effective amount of 3BP to the vagina so that sperm in contact with 3BP display aberrant, reduced or no motility, which are incompatible with fertilization, thereby preventing pregnancy. In one embodiment, a method includes topical application of 3BP to the penis in an amount effective to cause complete loss of motility of ejaculated sperm during sexual intercourse, thereby achieving contraception. In some examples, 3BP is administered as a spermicide in the forms of a jelly (gel), films, sponges, foams and dissolvable nanofiber or nanomesh. In some examples, condoms are coated with 3BP to achieve contraception.

As used herein, 3-bromopyruvate (3BP) is a synthetic brominated derivative of pyruvic acid. 3BP has been regarded as a glycolytic inhibitor, but it has been found to possess properties beyond its anti-glycolytic effect, which remain unknown. 3BP has the molecular formula of C₃H₃BrO₃. 3BP is also known as bromopyruvate, 3-bromopyruvic acid, or 3-bromo-2-oxopropanoic acid.

In some examples of inhibiting sperm motility so that sperm cannot fertilize eggs, thereby preventing pregnancy, the 3BP compositions of the present disclosure are intravaginally applied either directly or indirectly. For example, the 3BP compositions may be delivered intravaginally by applying as a lubricant, for example, on a condom or other device, including a sponge, cervical cap, tampon, diaphragm, or intrauterine device or by applying the composition as a suppository, douche, ovule, gel, or other controlled delivery device (e.g. dissolvable nanofiber or nanomesh). The 3BP compositions can be applied to any portion of the uterus by an intrauterine delivery device, such as those intrauterine devices (IUDs) known to those skilled in the art. Applicators known to the art, such as those currently used commercially to deliver spermicidal gels or anti-yeast compounds, may also be used to deliver the compositions.

An effective amount of the 3BP compositions is typically administered to reduce sperm motility by at least 50% within 5 seconds, or between 5 to 20 seconds or between 20 seconds to 60 seconds or between 60 seconds to 360 seconds, such as within 10 seconds, 20 seconds, 30 seconds, 40 seconds, 50 seconds, 60 seconds, 120 second, 180 seconds, 240 seconds or 360 seconds of contact between the 3BP composition and sperm. In some examples, an effective amount is to reduce sperm motility by at least 60% within 5 seconds, or between 5 to 20 seconds or between 20 seconds to 60 seconds or between 60 seconds to 360 seconds, such as within 10 seconds, 20 seconds, 30 seconds, 40 seconds, 50 seconds, 60 seconds, 120 second, 180 seconds, 240 seconds or 360 seconds of contact between the 3BP composition and sperm. In some examples, an effective amount is to reduce sperm motility by at least 70% within 5 seconds, or between 5 to 20 seconds or between 20 seconds to 60 seconds or between 60 seconds to 360 seconds, such as within 10 seconds, 20 seconds, 30 seconds, 40 seconds, 50 seconds, 60 seconds, 120 second, 180 seconds, 240 seconds or 360 seconds of contact between the 3BP composition and sperm. In some examples, an effective amount is to reduce sperm motility by at least 80% within 5 seconds, or between 5 to 20 seconds or between 20 seconds to 60 seconds or between 60 seconds to 360 seconds, such as within 10 seconds, 20 seconds, 30 seconds, 40 seconds, 50 seconds, 60 seconds, 120 second, 180 seconds, 240 seconds or 360 seconds of contact between the 3BP composition and sperm. In some examples, an effective amount is to reduce sperm motility by at least 85% within 5 seconds, or between 5 to 20 seconds or between 20 seconds to 60 seconds or between 60 seconds to 360 seconds, such as within 10 seconds, 20 seconds, 30 seconds, 40 seconds, 50 seconds, 60 seconds, 120 second, 180 seconds, 240 seconds or 360 seconds of contact between the 3BP composition and sperm. In some examples, an effective amount is to reduce sperm motility by at least 90% within 5 seconds, or between 5 to 20 seconds or between 20 seconds to 60 seconds or between 60 seconds to 360 seconds, such as within 10 seconds, 20 seconds, 30 seconds, 40 seconds, 50 seconds, 60 seconds, 120 second, 180 seconds, 240 seconds or 360 seconds of contact between the 3BP composition and sperm. In some examples, an effective amount is to reduce sperm motility by at least 95% within 5 seconds, such as between 5 to 20 seconds or between 20 seconds to 60 seconds or between 60 seconds to 360 seconds, such as within 10 seconds, 20 seconds, 30 seconds, 40 seconds, 50 seconds, 60 seconds, 120 second, 180 seconds, 240 seconds or 360 seconds of contact between the 3BP composition and sperm. In some examples, an effective amount is to reduce sperm motility by at least 98% within 5 seconds, or between 5 to 20 seconds or between 20 seconds to 60 seconds or between 60 seconds to 360 seconds, such as within 10 seconds, 20 seconds, 30 seconds, 40 seconds, 50 seconds, 60 seconds, 120 second, 180 seconds, 240 seconds or 360 seconds of contact between the 3BP composition and sperm. In some examples, an effective amount is to reduce sperm motility by at least 100% within 5 seconds, or between 5 to 20 seconds or between 20 seconds to 60 seconds or between 60 seconds to 360 seconds, such as within 10 seconds, 20 seconds, 30 seconds, 40 seconds, 50 seconds, 60 seconds, 120 second, 180 seconds, 240 seconds or 360 seconds of contact between the 3BP composition and sperm. In some examples, an effective amount is to reduce sperm motility by at least 95% within 5 minutes to 30 minutes, such as within 10 minutes, 15 minutes, 20 minutes, 25 minutes or 30 minutes. This effective amount, in the context of the methods of contraception described herein, is intended to mean that amount of 3BP, when administered to a mammal in need thereof, sufficient to effect inhibition of sperm-egg fertilization and embryo formation. In the case of its contraceptive properties, the effective amount of 3BP is that amount effective to decrease the possibility of sperm-egg fertilization, by reducing or inhibiting sperm motility and thus, inhibiting sperm-fertilizing capabilities. This amount of 3BP may be readily determined by one of skill in the art and dependent upon the route of administration. For example, the 3BP may be present, in an effective amount, in the form of a topical spermicide with about 1 to about 500 mM 3BP, or about 5 mM to 20 mM 3BP, or about 100 mM to 200 mM 3BP, for example, greater than 2 mM and less than 300 mM, such as 3 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 25 mM, 30 mM, 40 mM, 50 mM, 60 mM, 70 mM, 80 mM, 90 mM, 100 mM, 110 mM, 120 mM, 130 mM, 140 mM, 150 mM, 160 mM, 170 mM, 180 mM, 190 mM, 200 mM, 250 mM, 300 mM, 350 mM, 400 mM, 450 mM, or 500 mM.

In some embodiments, 3BP is administered to immobilize sperm by implanting a micro-device into the epididymis, where 3BP is released in a dose- and frequency-controlled manner. In this method, epididymal sperm are exposed to 3BP and thus, the ejaculated sperm will display no or abnormal motility, which is incompatible with fertilization. This can become a long-term, stable, and effective male contraceptive method if such a timed-release mini-implant is available. In this application the concentration of 3BP can be released at a frequency of once every 1 hour, 2 hours, 3 hours, 4 hours, 5 hours, 6hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 30 hours, 36 hours, 42 hours and 48 hours, and the local concentrations of 3BP can be about 1 to about 500 mM 3BP, or about 5 mM to 20 mM 3BP, or about 100 mM to 200 mM 3BP, for example, greater than 2 mM and less than 300 mM, such as 3 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 25 mM, 30 mM, 40 mM, 50 mM, 60 mM, 70 mM, 80 mM, 90 mM, 100 mM, 110 mM, 120 mM, 130 mM, 140 mM, 150 mM, 160 mM, 170 mM, 180 mM, 190 mM, 200 mM, 250 mM, 300 mM, 350 mM, 400 mM, 450 mM, or 500 mM.

Moreover, the “inhibition” of sperm-egg fertilization refers to the reduced occurrence of conception, i.e., sperm-egg fertilization, resulting in pregnancy relative to untreated individuals. Not being limited by any particular theory of the mechanism of action of the compositions described herein, it is believed that the inhibition of sperm motility by 3BP results in the inhibition of sperm-egg fertilization and effective contraception.

The compositions used in the methods described herein may include other agents that do not negatively impact or otherwise affect contraceptive effectiveness of the components of the composition, including glycolytic inhibitors (such as 2-deoxyglucose, lonidamin and oxythiamine), antimicrobial agents, additional sperm-function inhibitors, or derivatives of 3BP. For example, solid, liquid or a mixture of solid and liquid pharmaceutically acceptable carriers, diluents, vehicles, or excipients may be employed in the pharmaceutical compositions. Suitable physiologically acceptable, substantially inert carriers include water, a polyethylene glycol, mineral oil or petrolatum, propylene glycol, hydroxyethylcellulose, carboxymethyl cellulose, cellulosic derivatives, polycarboxylic acids, linked polyacrylic acids, such as carbopols; and other polymers such as poly(lysine), poly(glutamic acid), poly(maleic acid), poly(lactic acid), thermal polyaspartate, and aliphatic-aromatic resin; glycerin, starch, lactose, calcium sulphate dihydrate, terra alba, sucrose, talc, gelatin, pectin, acacia, magnesium stearate, stearic acid, syrup, peanut oil, olive oil, saline solution, and the like. In some embodiments, dissolvable nanofiber or nanomesh can be used that can act as drug carrier with the capability of timed release.

The pharmaceutical compositions described herein useful in the methods of the present disclosure may further include diluents, fillers, binding agents, moisturizing agents, preservatives, acids, and other elements known to those skilled in the art. For example, suitable preservatives are well known in the art, and include, for example, methyl paraben, propyl paraben, butyl paraben, benzoic acid and benzyl alcohol.

The compositions used in the methods of the present disclosure may be employed in any form suitable for effective sperm motility inhibition. For example, the compositions of this present disclosure could be in various forms known to the art, including liquid form or in lotion form, either oil-in-water or water-in-oil emulsions, in aqueous gel compositions, in the form of foams, films, sprays, ointments, pessary, suppository, capsules, tablets, jellies, creams, liposomes or in other forms embedded in a matrix for the slow or controlled release of the biologically active material to the skin or surface onto which it has been applied or in contact. In some examples, the compositions of the present invention are aqueous compositions. In some examples, the compositions are aqueous gel compositions. Dissolvable drug-loaded nanofiber can be the carrier as well. Mini- or micro-implant carrying 3BP for timed release can be an alternative means of delivery.

The disclosure is further illustrated by the following non-limiting Example.

### EXAMPLE

### This Example Demonstrates That 3BP is a Spermicide/Sperm Immobilizer and Can be Used as a Topical Contraceptive Agent

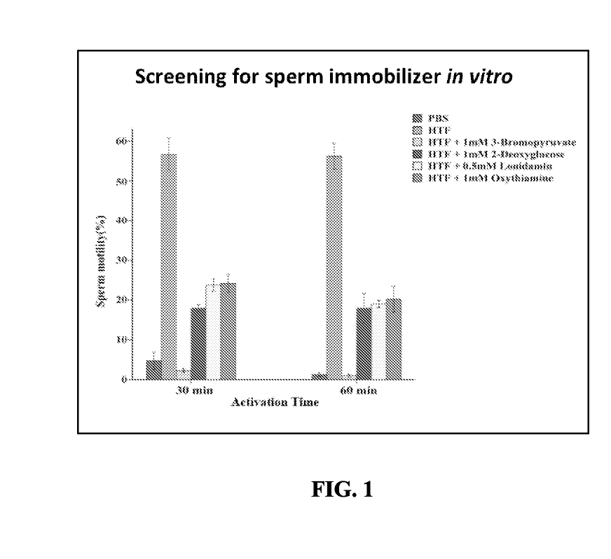

Sperm motility requires energy, the sources of which include both glycolysis and mitochondrial activity. The effects of four glycolytic inhibitors (3-bromopyruvate, 2-deoxyglucose, lonidamin and oxythiamine) on sperm motility were evaluated. First mouse cauda epididymal sperm were collected into PBS, HTF (sperm-activating solution), HTF supplemented with 1 mM 3-Bromopyruvate, HTF containing 1 mM 2-Deoxyglucose, HTF with 0.5 mM Lonidamin and HTF with 1 MM Oxythiamine. Epididymal sperm can gain partial motility in PBS, but will never develop hyperactivated motility, which is required for sperm to be competent to fertilize eggs. On the other hand, when epididymal sperm are incubated in human tubular fluid (HTF) medium, which contains all of the sperm motility-activating factors, sperm will quickly develop hyperactivated motility and the motility peaks within 30 minutes and can last at least for several hours at 37° C. In the present studies, mouse cauda epididymal sperm were incubated in PBS, HTF and HTF supplemented with the 4 compounds for 30 min and 60 min. Sperm motility was then measured by Computer-Assisted Sperm Analysis (CASA). The results are shown in FIG. 1. Although all 4 compounds appeared to inhibit the development of sperm motility, 3-bromopyruvate (3BP) stood out as the most efficient one that almost completely blocked the development of sperm motility. These studies indicate that in the presence of 3BP (at 1 mM), sperm cannot develop motility.

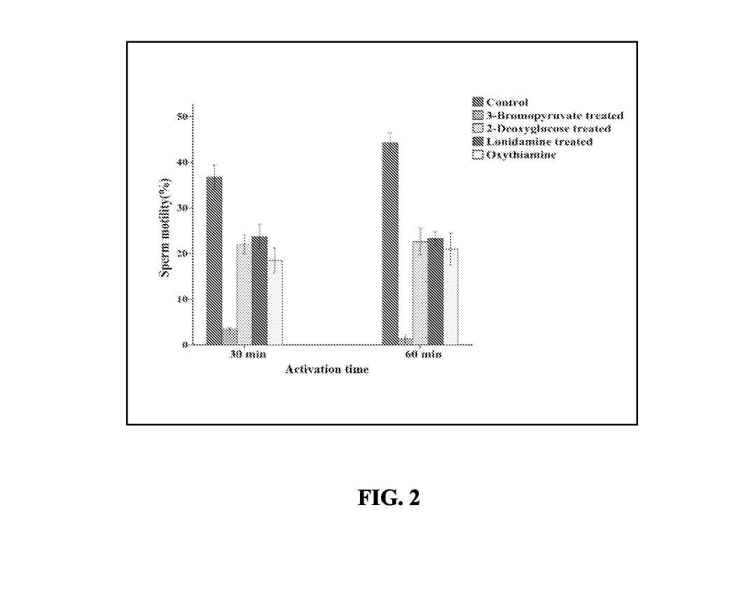

Studies were then performed to determine if sperm-immobilizing effects of 3BP were permanent or transient. Mouse cauda epididymal sperm were collected into PBS (control), PBS containing 1 mM 3BP, PBS containing 1 mM deoxyglucose, PBS with 0.5 mM lonidamine and PBS with 1 mM oxythiamine, followed by incubation at 37° C. for 5 min. The compounds were then washed away with PBS and then the treated sperm were incubated in HTF medium for 30 minutes and 60 minutes, followed by measurement of sperm motility by CASA. FIG. 2 demonstrates that a short exposure (5 minutes) of sperm to 3BP or one of the other 3 compounds will permanently impair motility development. Again, 3BP had the strongest effects. Once sperm were exposed to 3BP, they could not develop motility no matter how long they were incubated in motility activating (i.e. HTF) medium. These studies indicate that the sperm motility-abolishing effect of 3BP was permanent. Further, once exposed, sperm will never be able to develop motility. These studies demonstrate that regardless when 3BP becomes in contact with sperm, either in their inactive (e.g., in the epididymis) or active (e.g., in the female reproductive tract) status, sperm motility is lost as soon as they are exposed to 3BP.

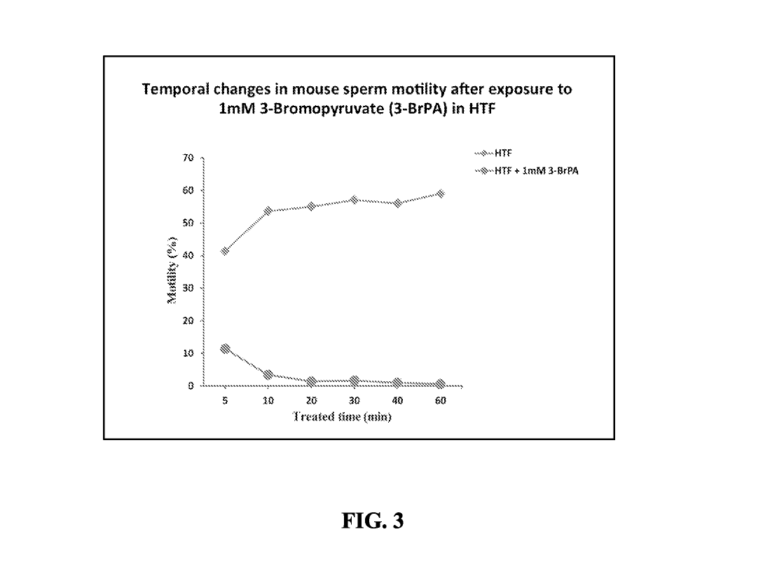

The first two studies indicated that 3BP could immobilize sperm during 30-60-minute incubation in vitro. To determine how quickly 3BP could immobilize sperm, mouse cauda epididymal sperm were collected into HTF and HTF supplemented with 1 mM 3BP, respectively. Sperm were then incubated at 37° C. for 5, 10, 20 30, 40 and 60 min, respectively. Sperm motility was completely lost within 10 minutes at 1 mM concentration and the inhibitory effect appeared to be permanent (see FIG. 3).

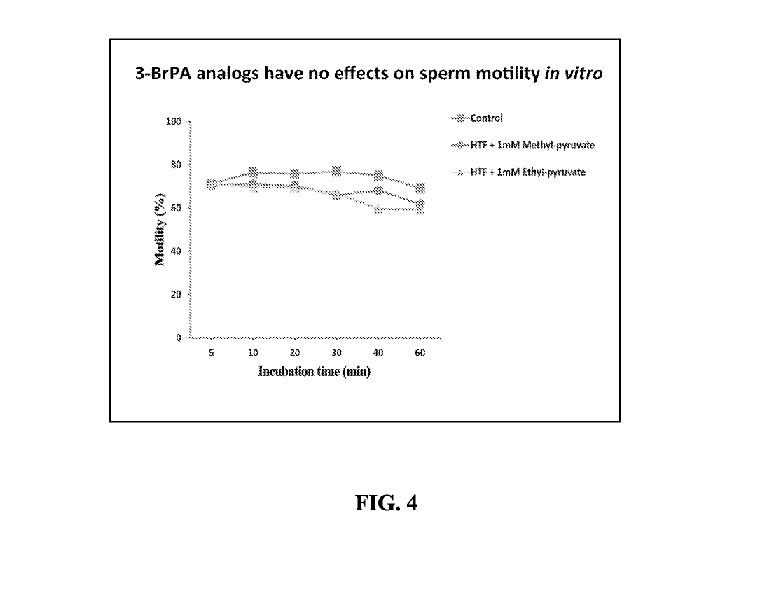

Given that 3BP could immobilize sperm, it was determined if analogs of 3BP could have the same effect. Four 3BP analogs, including methyl-pyruvate, ethyl-pyruvate, isoamyl-pyruvate and propyl-pyruvate, were tested to determine if they also had the inhibitory effect on sperm in vitro. In the first study, the effects of methyl-pyruvate and ethyl-pyruvate on sperm motility were evaluated (FIG. 4A). Mouse epididymal sperm were collected into HTF (control), HTF containing 1 mM Methyl-pyruvate and HTF with 1 mM ethyl-pyruvate, respectively. After incubation at 37° C. for 5, 10, 20, 30, 40 and 60 min, sperm motility was measured using CASA. None of the treatments resulted in inhibition of sperm motility in vitro. Similarly, the effects of isoamyl-pyruvate and propyl-pyruvate on sperm motility in vitro were evaluated (FIG. 4B) and again, such treatments had no effect on sperm motility in vitro.

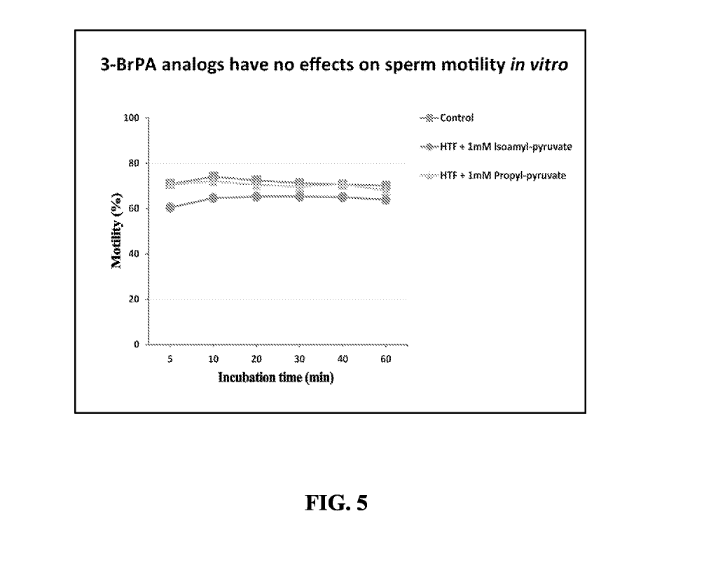

To determine if sperm exposed to 3BP could still be activated in HTF, mouse sperm from the cauda epididymis were collected into PBS (control) and PBS containing 1 mM 3-BrPA, respectively. Then sperm were incubated in 37° C. for 1, 5, 10, 20 and 30 min, respectively. To remove 3BP, the sperm were washed twice with PBS. The sperm were then transferred to the HTF medium and activated for 30 min (FIG. 5, A) and 60 min (FIG. 5, B), followed by measurement the sperm motility using CASA. No motility was developed in sperm exposed to 3-BrPA for 1-30 min. The data indicate that the sperm-immobilizing effects of 3BP are permanent or irreversible.

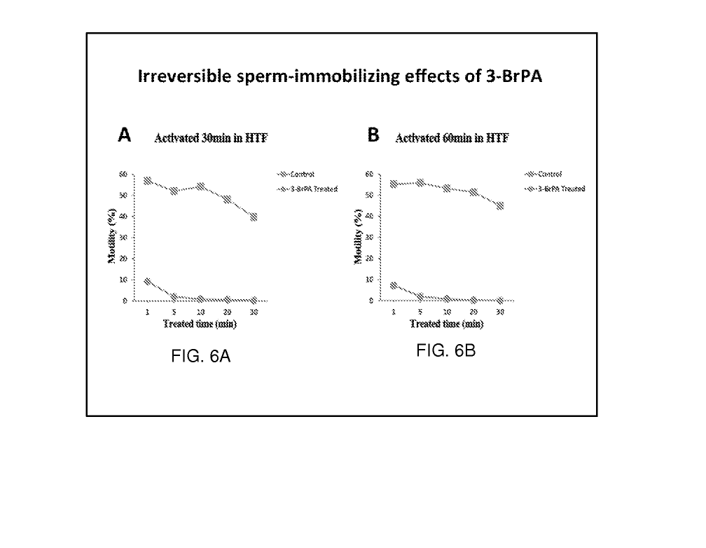

In normal men in their reproductive ages, ˜30-40% of sperm in human ejaculates are immotile or display weak motility. A ‘swim-up” procedure allows motile sperm to be enriched. To see the potential of 3BP in suppressing sperm motility, the swim-up procedure was first performed to allow the collection of motile sperm and then the motile human sperm were exposed to 3BP to determine if their motility could be abolished. The human semen was left at room temperature for 30 minutes for liquidification and then 2˜3 ml pre-warmed HTF medium were added followed by centrifugation at 1,500 rpm for 5 min. The sperm pellets were resuspended in 1 ml pre-warmed HTF medium and then incubated at 37° C. in anincubator for 60 min. Motile sperm will “swim up” and stay in the upper portion of the medium, in which motile sperm are enriched in. The “swim-up” sperm were collected, 3BP added at a concentration of 1 mM, and the sperm were incubated for 1, 5, 10, 20 and 30 min at 37° C., respectively. Sperm motility was then measured using CASA (FIG. 6). These studies demonstrate that 3BP can abolish motile human sperm with 1-5 minutes.

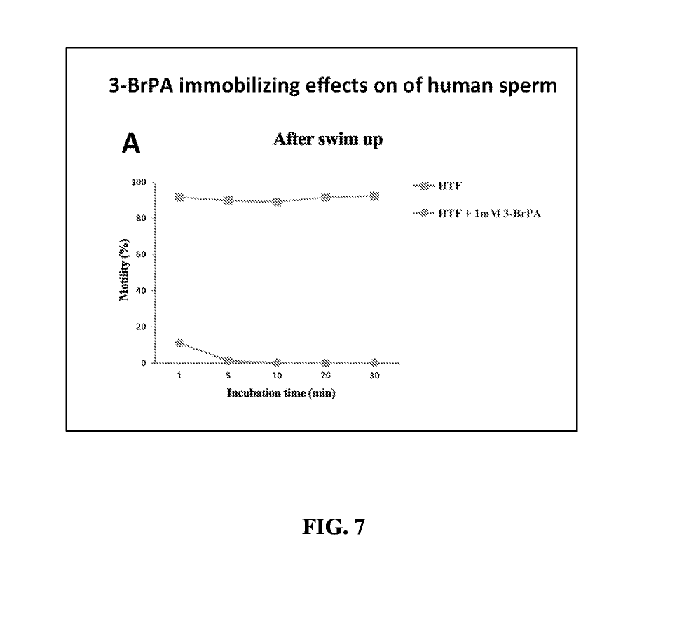

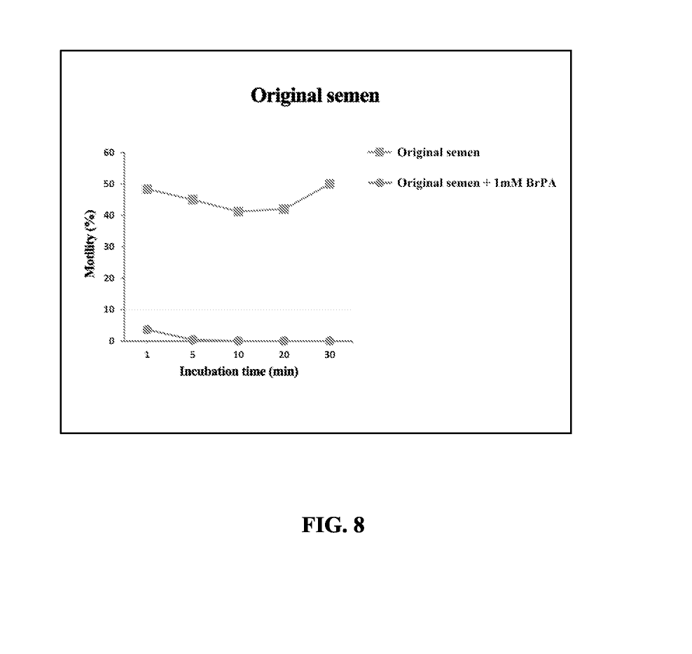

To evaluate the effect of 3BP on sperm motility in human semen, human semen was liquidified under room temperature and 3BP was directly added into the liquidified semen at a concentration of 1 mM followed by incubation at 37° C. for 1, 5, 10, 20 and 30 minutes (FIGS. 7 and 8). These studies indicate that 3BP can completely abolish human sperm motility within 5 minutes of contact.

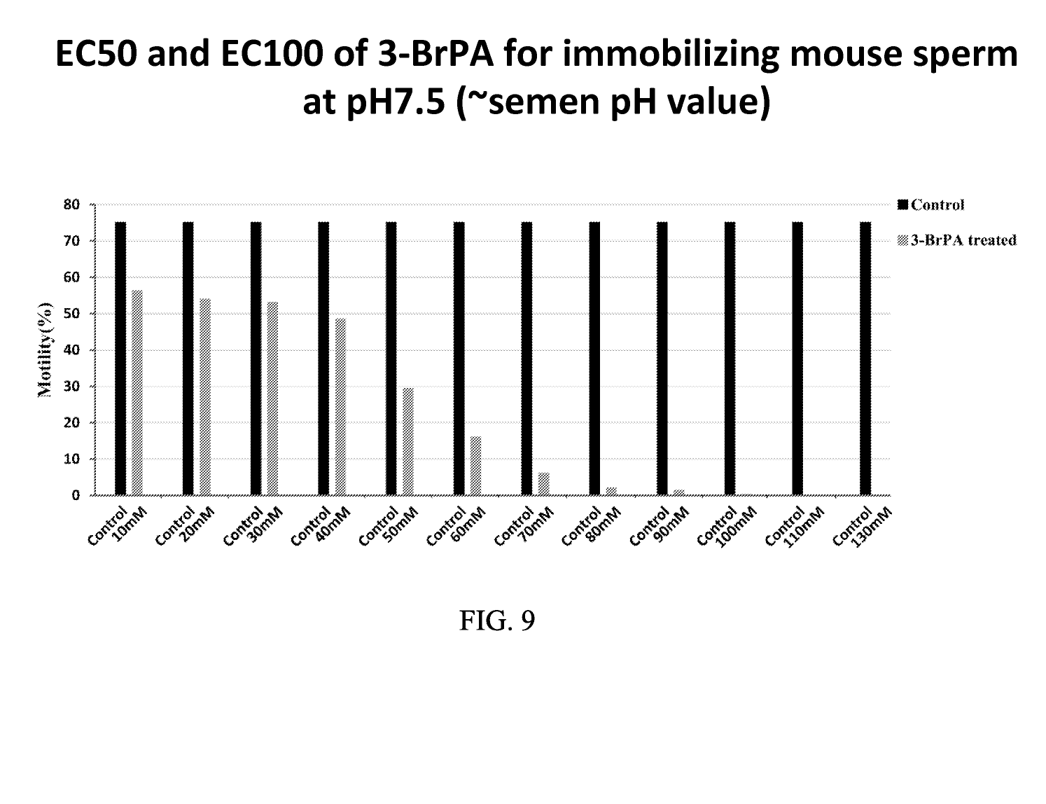

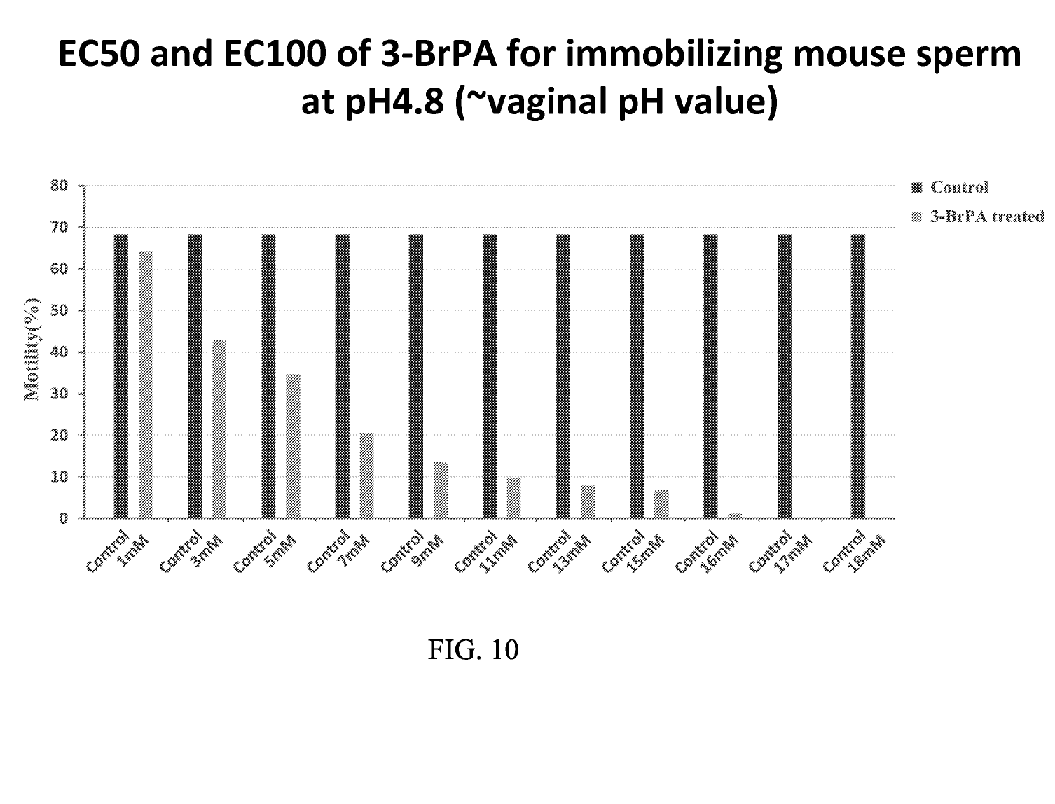

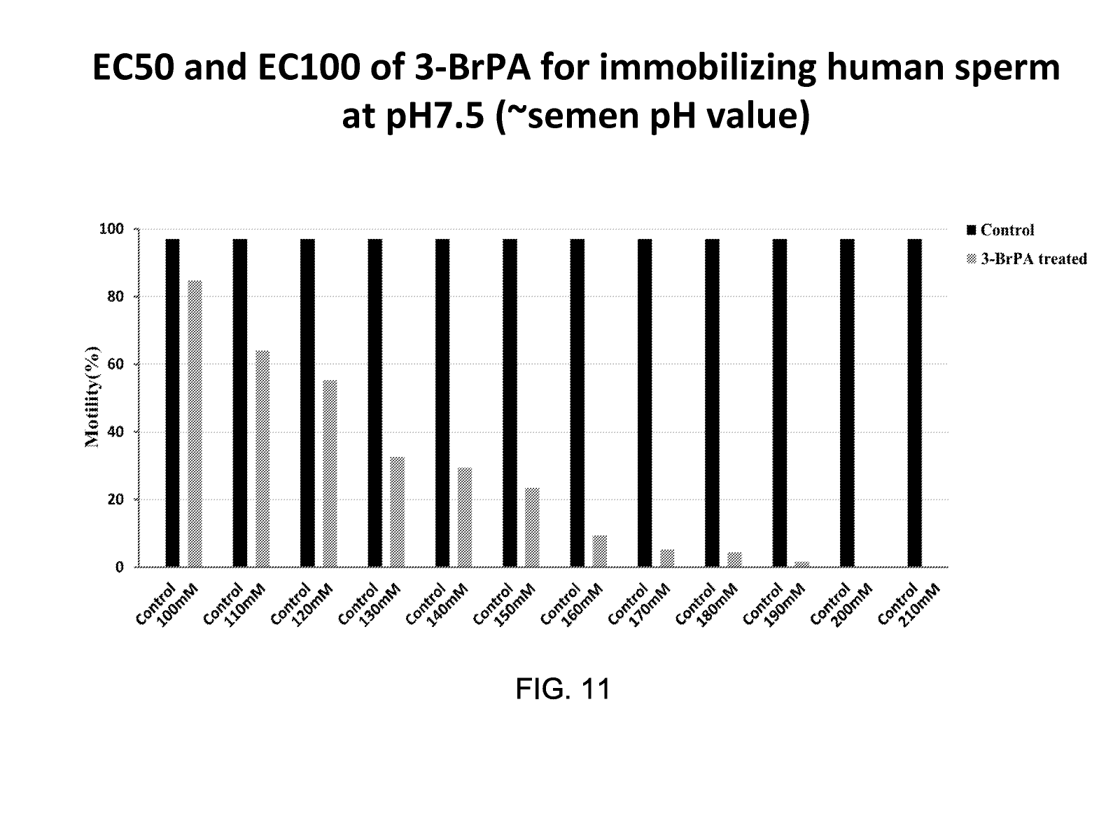

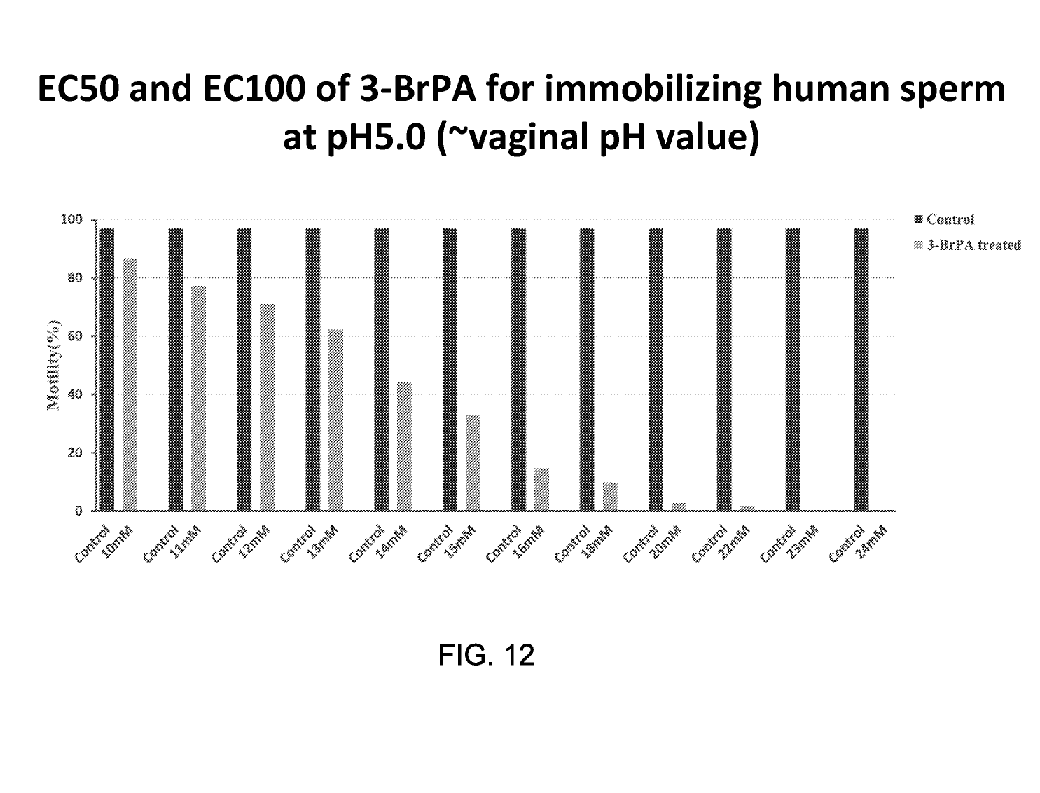

The concentrations at which 50% and 100% of the total motile sperm can be “killed/immobilized”within 20 seconds of contact have been defined as EC50 and EC100, respectively (FIGS. 9, 10, 11 and 12). To determine at what concentrations 3BP can block the motility of 50% and 100% of the total motile sperm, mouse epididymal sperm were collected into PBS (pH 7.5) and then transferred to pre-warmed (37° C.) HTF medium containing serial dilutions of 3BP (pH 7.5) at concentrations ranging from 10 to 130 mM with a 10 mM increment. Motility was measured using CASA upon 20 seconds of contact with 3BP. Sperm motility was found to be decreased to half of the control samples after sperm were exposed to 40˜50 mM 3BP for 20 sec and completely lost their motility after treatment with 100 mM 3BP for 20 sec. Therefore, the 3BP EC50 is ˜40˜50 mM and EC100 is ˜100 mM for mouse epididymal sperm at pH 7.5 (FIG. 9). Similarly, the EC50 and EC100 of 3BP in immobilizing human sperm were determined. Human “swim-up” sperm were added to HTF containing 3BP at increasing concentrations ranging from 100 mM to 210 mM at pH 7.5. Sperm motility was measured upon 20 seconds of contact using CASA. It was determined that EC50 of 3BP in human sperm is ˜120-130 mM, and EC100 is ˜200 mM at pH 7.5 (FIG. 11). pH 7.5 was chosen because it represents the normal pH value of semen in both mice and humans. However, the vaginal environment tends to be acidic (˜pH 5.0) and the effectiveness of 3BP in immobilizing sperm was further evaluated when pH was set at ˜5.0 (FIGS. 10 and 12). Interestingly, at ˜pH 4.8, EC50 and EC100 of 3BP for mouse sperm were determined to be ˜5 mM and ˜17 mM, respectively (FIG. 10). EC50 and EC100 of 3BP for human sperm were determined to be 14 mM and 23 mM, respectively (FIG. 12).

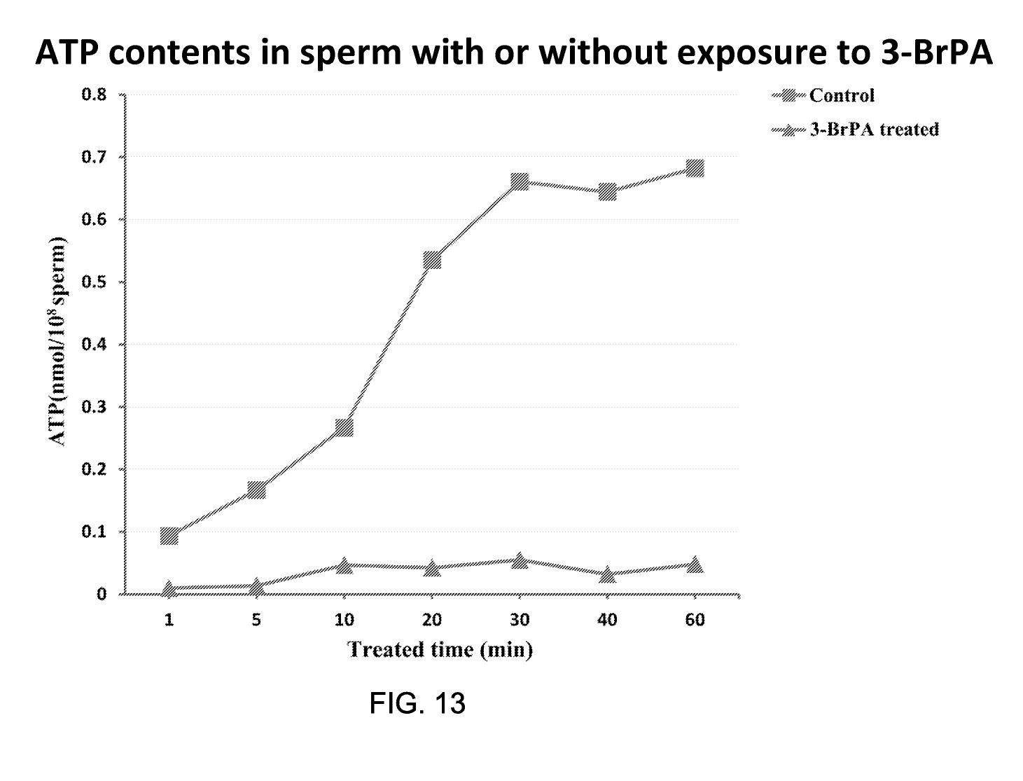

The ATP production in sperm was measured with or without exposure to 1 mM 3BP during 60 min activation by HTF. Sperm ATP contents were measured using a Luciferase Bioluminescence Assay kit (FIG. 13). These studies demonstrate that 3BP can effectively block sperm ATP production, which explain its motility-inhibiting effects. However, such a drastic suppression cannot be explained solely by its anti-glycolytic property because glycolysis provides only partial energy sperm motility requires. Moreover, none of the other 3 known glycolytic inhibitors tested exhibited a total block of sperm motility and ATP production as 3BP does.

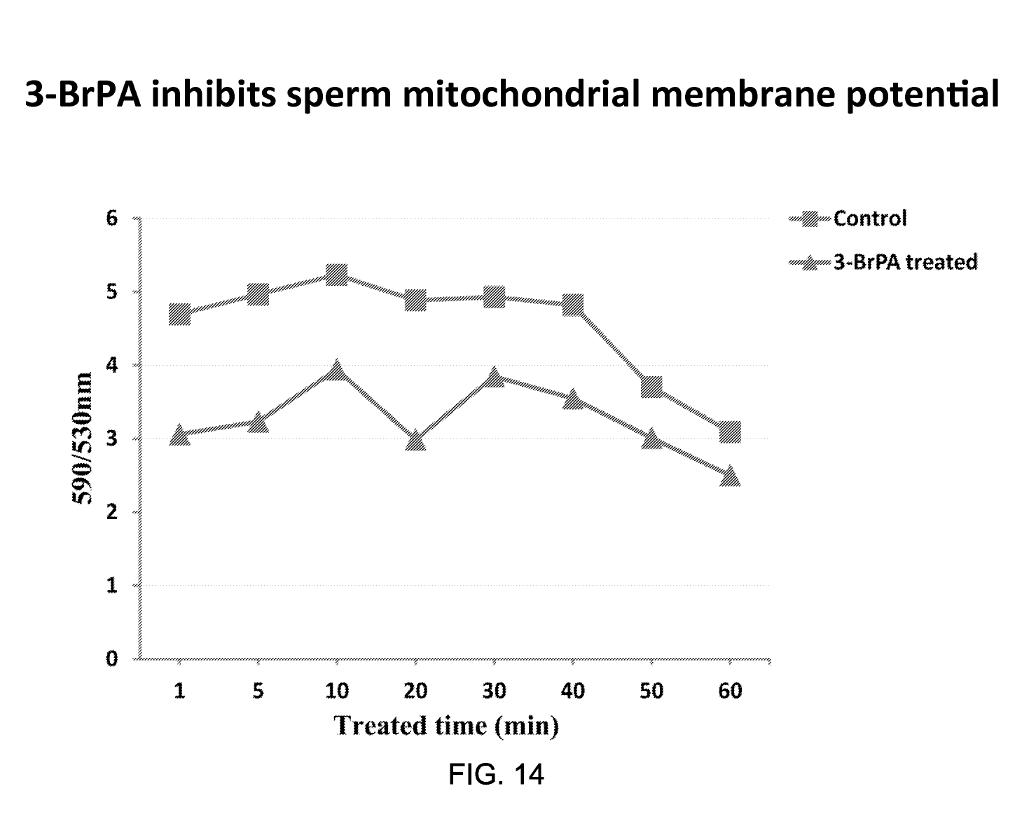

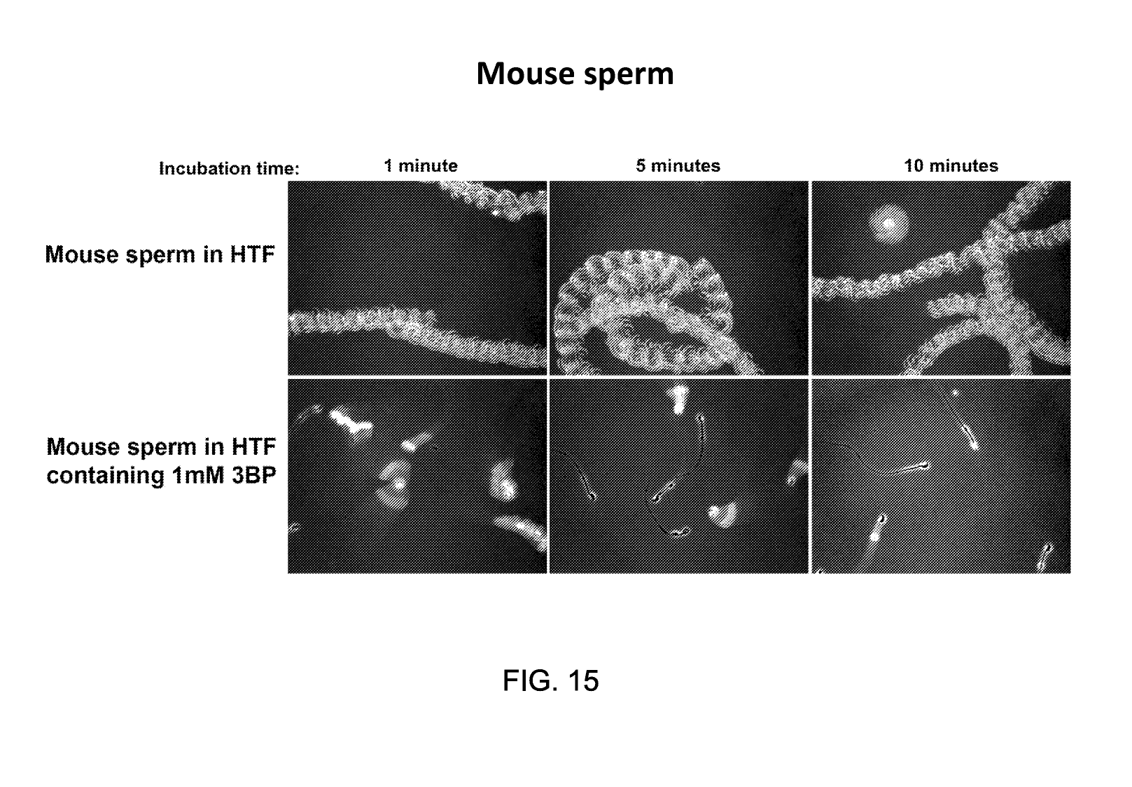

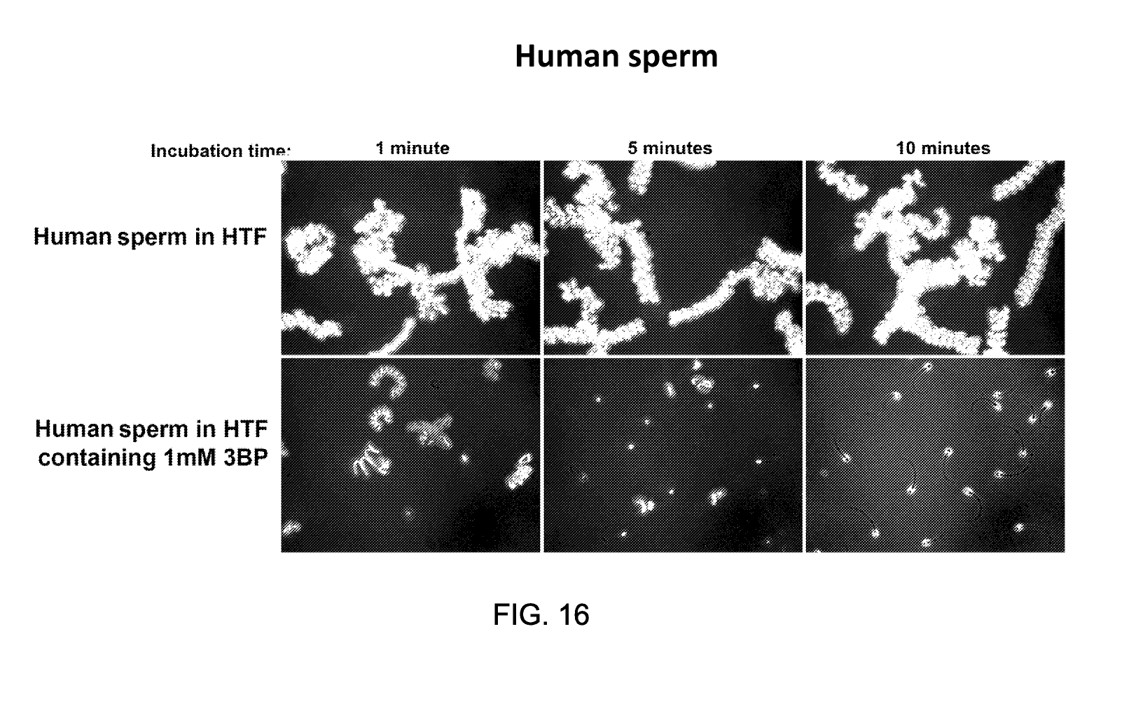

Whether 3BP could block mitochondrial activity in sperm was further examined because mitochondria also contribute energy production in sperm (FIG. 14). By measuring the sperm mitochondrial membrane potential using JC-1 assays, it was found that indeed 3BP could inhibit the mitochondrial membrane potential, indicating that its immobilizing effects in part involve its ability to decrease mitochondrial activity. In control sperm trajectory tracking assays, the cauda epididymal sperm were collected into the HTF medium and incubated for 1, 5, 10 and 20 min (FIG. 15). In 3BP-treated sperm trajectory tracking assays, the cauda epididymal sperm were collected HTF+1 mM 3-BrPA and incubated for 1, 5, 10 and 20 min (FIG. 15). In control human sperm trajectory tracking assays, the human “swim-up” sperm were incubated HTF for 1, 5, 10, and 20 minutes (FIG. 16). In 3BP-treated human sperm movie clips, the human “swim-up” sperm were incubated in HTF+1 mM 3-BrPA for 1, 5, 10 and 20 minutes (FIG. 16). Further, the histology of the vagina and uterus in female mice that received daily intravaginal application of 3BP (200-400 ul lubricant containing 20 mM 3BP) for 3 months was accessed. No discernible pathological changes were observed, indicating that 3BP can be used intravaginally on a daily basis.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. I therefore claim as my invention all that comes within the scope and spirit of these claims.

1. A method of contraception, comprising: administering a composition comprising an effective amount of 3-bromopyruvate (3BP) to inhibit sperm motility, thereby inhibiting sperm-egg fertilization and achieving contraception.

2. The method of claim 1, wherein administering comprises topical application of 3BP to a subject's penis in an amount effective to inhibit the motility of ejaculated sperm during sexual intercourse, thereby causing permanent loss of motility and thus, achieving contraception.

3. The method of claim 1, wherein administering comprises administering to a subject's vagina an effective amount of 3BP effective to inhibit sperm motility.

4. The method of claim 1, wherein an effective amount of 3BP comprises administering an amount of 3BP capable of inhibiting at least 50% of sperm motility within 5 seconds to 360 seconds of contact between the 3BP composition and sperm.

5. The method of claim 4, wherein an effective amount of 3BP comprises an amount of 3BP capable of inhibiting at least 95% of sperm motility within 5 seconds to 360 seconds of contact between the 3BP composition and sperm.

6. The method of claim 1, wherein the composition comprises greater than 1 mM 3BP but less than 500 mM 3BP.

7. The method of claim 6, wherein the composition comprises between 10 mM and 500 mM 3BP.

8. The method of claim 6, wherein the composition comprises between 100 mM and 500 mM 3BP.

9. The method of claim 1, wherein 3BP is administered in the form of spermicide, such as a jelly (gel), films, sponges, foams, nonofiber/nanomesh and intra-epididymal micro- or mini-implants.

10. The method of claim 2, wherein 3BP is administered by a condom lubricated or coated with 3BP.

11. The method of claim 1, wherein the composition comprises a pharmaceutical carrier.

12. The method of claim 1, wherein the composition further comprises one or more active ingredients, such as one or more antimicrobial agents and/or one or more additional contraceptive agents.

13. The method of claim 12, wherein the one or more additional contraceptive agents comprise one or more glycolytic inhibitors.

14. The method of claim 1, wherein the composition consists essentially of 3BP and is administered to provide contraception, antimicrobial and anti-cancer activity.
